(19) United States
(12) Patent Application Publication
(10) Pub. No.: US 2011/0040261 A1
(43) Pub. Date: Feb. 17, 2011

(54) DEVICE SUITABLE FOR ADMINISTERING LIQUID SUBSTANCES INTO THE HUMAN BODY

(76) Inventors: Dario Petrone, Pignola (IT); Mirco Cavicchioli, Modena (IT)

Correspondence Address: Gregory A. Nelson;Novak Druce & Quigg LLP, 525 Okeechobee Blvd, Suite 1500, West Palm Beach, FL, 33401, US

(21) Appl. No.: 12/676,799

(22) PCT Filed: Sep. 8, 2008
(86) PCT No.: PCT/IT2008/000572

§ 371 Date: Oct. 29, 2010

(30) Foreign Application Priority Data
Sep. 7, 2007 (IT) BO2007A000611

(51) Int. Cl. A61M 25/00

(52) U.S. Cl. 604/264

(57) ABSTRACT

Object of the present invention is an entirely closed circuit apparatus for the administration of anesthetics, and/or other liquids into the human body during operation sessions. Such apparatus allows dosing an accurate amount of drug during each operation session and to each single patient without drug wastage and without using syringes with needles.

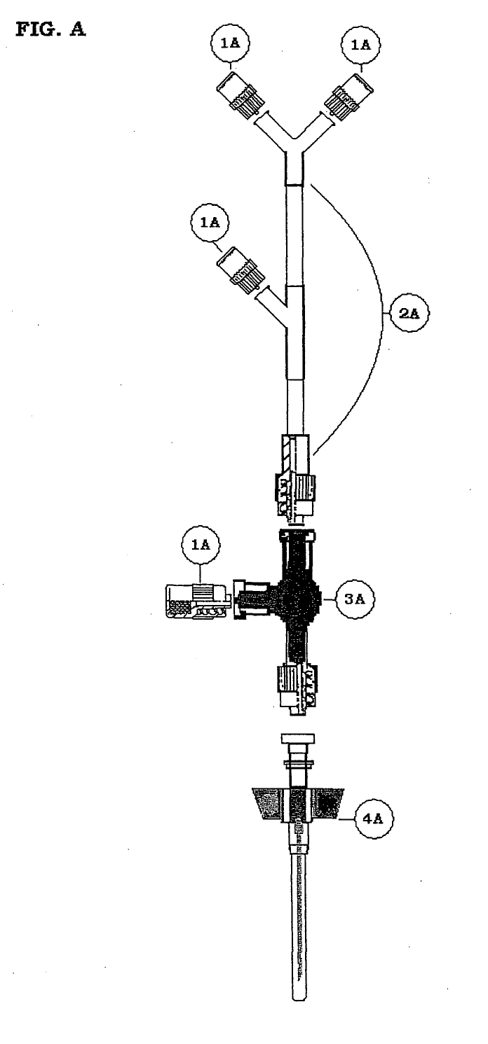

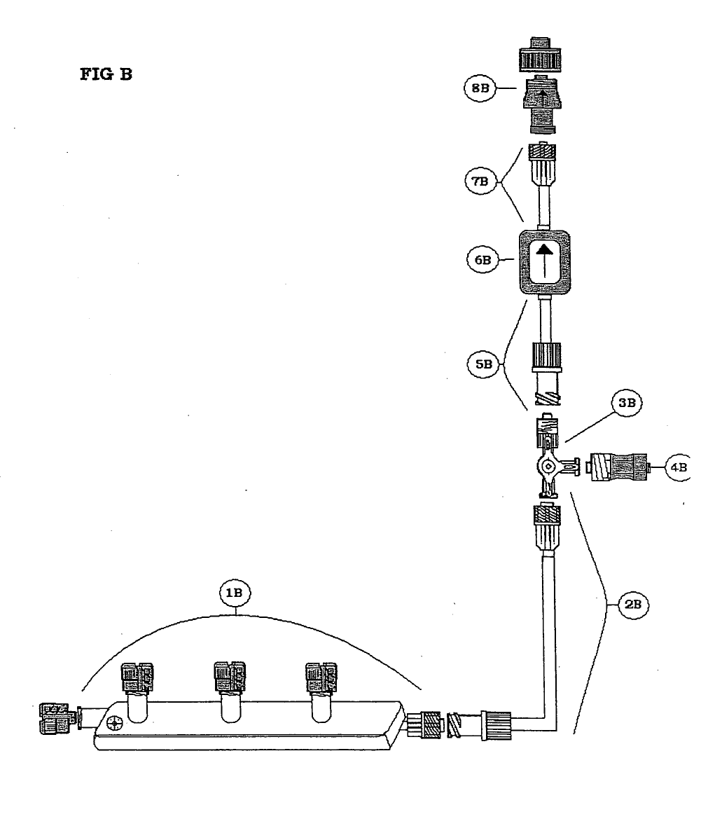

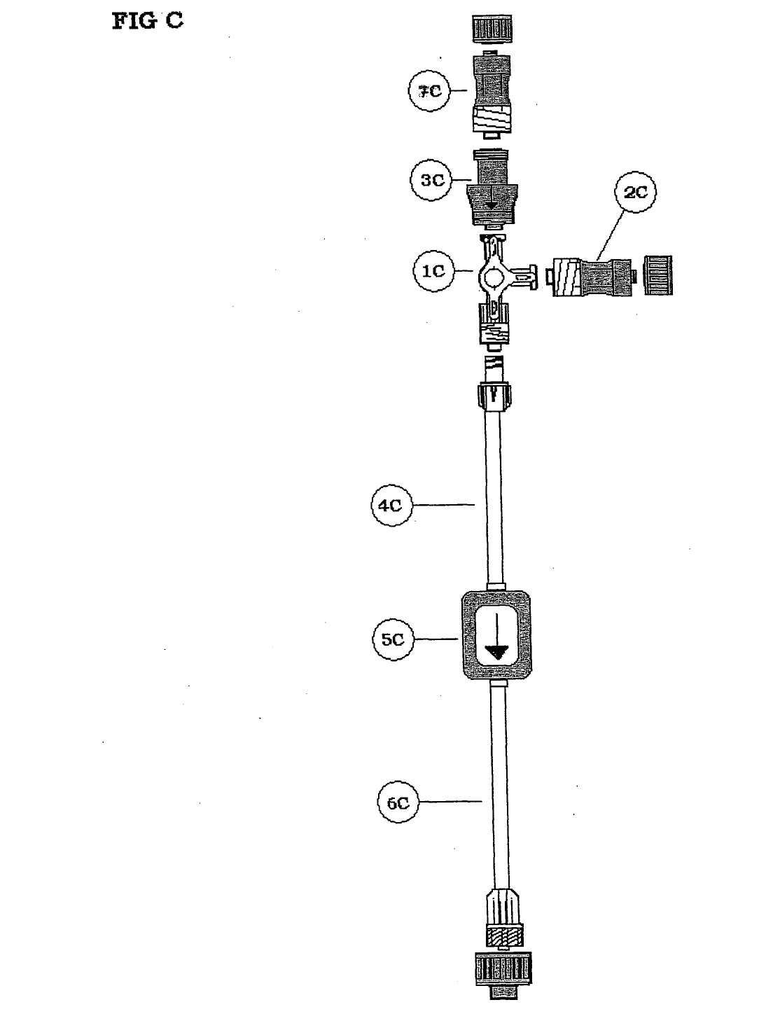

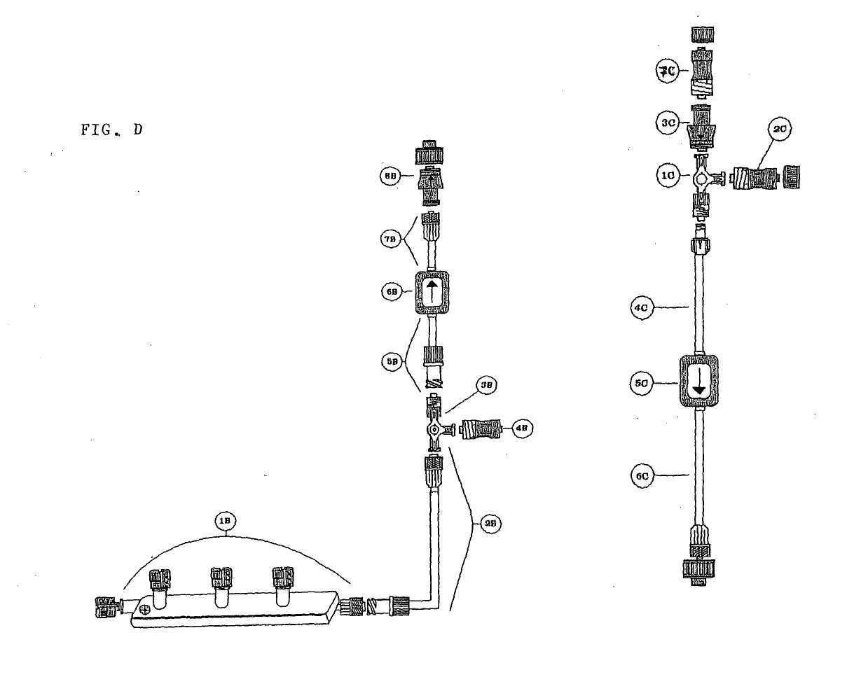

### FIELD OF THE INVENTION

The present invention refers to an apparatus for the administration of anaesthetics or other liquids into the human body. Used for dosing the required drugs without wastage, such apparatus is assembled in such a manner to create a closed circuit minimising risks related to possible contaminations and/or infections.

### PRIOR ART

Apparatus for administering anaesthetics or other liquids into the human body currently available in the market are made up of infusion set/extensions assembled to devices able to be perforated by needles which, applied to the syringes, allow the infusion of drugs; the entire assembly is connected to the cannula needle which is inserted in the patient's vein. Such apparatus, assembled on the spot, including the syringes containing, the drugs, is disposed of at the end of the process.

This conventional apparatus, described in fig. A, has the following drawbacks:

compulsory use of syringes provided with needles which may cause inadvertent pricking of the operators;

risks related to air embolism, as devices for discharging air possibly present in the channel are not used;

risks of bacteriological contamination due to non-closed circuits, absence of check valves;

waste of drugs, as they cannot be used for the subsequent interventions.

### SUMMARY

An object of the present invention is an apparatus for administering anaesthetics of other liquids or solutions, comprising at least one system for simultaneous or deferred administration, having a plurality of syringes applied to a channel of various lengths which might bear inserted in its extension a system for extemporaneous application and a system for evacuating any air that might have been formed in the channel connected at whose end is a unidirectional apparatus for the flow of the anaesthetics or liquids or solutions; next, applied to the previous one, is an apparatus provided with a diverter applied onto which is one or more devices for quick loading/drawing, connected to a channel of varying length, to be in turn connected to the patient.

### LIST OF DRAWINGS

The art prior to the invention is outlined in the example of the attached figure A described below:

Fig. A shows the insertion points (injection point) (1A) used to administer anaesthetics or other liquids by means of needles, connected to an infusion set/extension (2A) and to the diverter (tap) (3A); the entire assembly is then connected to a cannula needle for the insertion into the patient's vein (4A).

The object of the invention is outlined in the examples of the attached figures B, C and D described in details below:

Fig. B shows the point of insertion (ramp) (1B) of the devices containing anaesthetics or solutions or drugs fitted by means of a pipe (2B) to a diverter (tap) (3B) joined to an accessory (4B) which allows loading/drawing liquids without using needles, the diverter (tap) (3B) is fitted by means of a pipe (5B) to an apparatus (filter) (6B) for eliminating air, downstream fitted by means of a pipe (7B) to a unidirectional apparatus (check valve) (8B).

Fig. C shows a diverter (tap) (1 C) with the accessory (2C) which allows loading and/or drawing liquids without using needles; a unidirectional apparatus (check valve) (3C) able to force the liquids to flow in a single direction is inserted in front of the accessory. The diverter (tap) (1C) is fitted by means of a pipe (4C) to an apparatus (filter) (5C) able to eliminate any air present in the channel, such apparatus (filter) (5C) is downstream fitted by beans of a pipe (6C) to the terminal fitting, directed to the patient through a needle or catheter.

Figure D shows the apparatus of figures B and C as a whole.

### DETAILED DESCRIPTION OF THE INVENTION

Figures B, C and D show the composition object of theinvention. In particular, figure B shows the point of insertion (ramp) (1B) of the devices containing anaesthetics or other liquids which, when applied, allow the simultaneous or deferred draining of several substances; the entire assembly is fitted by means of a pipe (2B) to a diverter (tap) (3B), permanently joined to which is an accessory (4B) which allows loading and/or drawing liquids, without the use of needles; during the loading step, the accessory (4B) acts as a further loading point for liquids intended for the patient; during the drawing step (possibly supported by suctioning pumps or other equivalent systems) the accessory acts as an outlet, useful for collecting aliquots of the fluid flowing in the pipe (2B) for monitoring, analysis purposes etc. The diverter (tap) (3B) is fitted by means of a pipe (5B) to a device (filter) (6B) able to eliminate any air present in the channel, such device being fitted to a unidirectional apparatus (check valve) (8B) which forces the liquids to flow only in a single direction.

The apparatus shown in figure B can be connected to the apparatus shown in figure C; the connection is performed between the afore described element (8B) and element (7C). Element (7C) is a connection element having the characteristic of being disconnectable from the rest of the apparatus without leaving any external liquid traces; such function is also performed by the afore described introduction/ drawing accessories (per se known); thus, the element (7C) may also be a further introduction/drawing accessory like the ones described elsewhere in the present application.

The apparatus illustrated in Fig. C comprises a diverter (tap) (1C) permanently fitted to which is one or more accessories (2C) allowing introducing/drawing liquids without using needles; the function of these accessories is identical to the one described for accessory (4B). A unidirectional apparatus (check valve) (3C) is permanently fitted upstream to the one or more accessories (2C) able to force the liquids to flow in a single direction. The diverter (tap) (1C) is fitted by means of a pipe (4C) to an apparatus (filter) (5C) adapted to eliminate possible air present in the channel; such apparatus (filter) (5C) is fitted downstream by means of a pipe (6C) to the terminal fitting. Lastly, the terminal fitting can be connected to the needle or catheter for the insertion into the patient's vein.

The apparatus described in Fig. B connected to the apparatus described in Fig. C represents the object of the invention and has the characteristic of being useable repeatedly up to the complete consumption of the liquids held in the connected devices applied to the point of insertion (ramp) (1B).

In order to be used again, the apparatus described in Fig. B must be connected to a second, then a third, and so forth, apparatus described in Fig. C. After each procedure, the apparatus in Fig. C remains connected to the patient in such a manner to continue other treatments.

The possibility to split the present apparatus into the two portions corresponding to figures B and C represents an important characteristic of the invention, as it allows the operators to continue the therapy using only the portion in figure C for a suitable period of time, preferably up to 96 hours, without requiring the replacement of the apparatus with others and minimising the possibility of contamination.

1. Device with an entirely closed circuit for administering anaesthetics or other solutions or liquids, characterised in that it has at least one main channel made up of: a point (1B) for inserting devices containing said anaesthetics or other solutions or liquids, fitted by suitable means to a diverter (3B), permanently joined to which is one or more accessories (4B) allowing loading/drawing liquids without using needles; the diverter (3B) is fitted by suitable means to an apparatus (6B) able to eliminate any air present in the channel, such apparatus (6B) being fitted by suitable means to a unidirectional device(8B) which forces the solutions or liquids to flow in a single direction; the apparatus (8B) is fitted through an element connectable/disconnectable without spilling the liquid to the external (7C), to a unidirectional apparatus (3C) which forces the solutions or liquids to flow in a single direction, and then to a diverter (1C), permanently fitted to which is one or more accessories (2C) which allow loading/drawing liquids without using needles; the diverter (1C) is fitted by suitable means to an device(5C) able to eliminate any air present in the channel, and the apparatus (5C) is fitted to an terminal fitting.

2. Device according to claim 1, characterised in that it allows the users to optimise the intake of drugs without wastage.

3. Device according to claim 1-2, characterised in that the first portion, made up of elements (1B) to (8B) and the second portion, made up of elements (7C) to the end fitting, can be separated, allowing the operators to continue the therapy using the second portion alone.
